(19) United States
(12) Patent Application Publication
(10) Pub. No.: US 2023/0028563 A1
(43) Pub. Date: Jan. 26, 2023

(54) Assembly for Optically Preconditioning an Optically Activable Biological Sample

(71) Applicant: ALBERT-LUDWIGS-UNIVERSITAET FREIBURG, Freiburg (DE)

(72) Inventors: Kathrin Brenker, Freiburg (DE); Luis Köbele, Freiburg (DE)

(21) Appl. No.: 17/790,813

(22) PCT Filed: Jan. 8, 2021
(86) PCT No.: PCT/EP2021/050252

§ 371 Date: Jul. 5, 2022

(30) Foreign Application Priority Data
Jan. 9, 2020 (DE) 10 2020 200 193.6

(51) Int. Cl. B01L 3/00; C12Q 1/24; G01N 15/14

(52) U.S. Cl. CPC B01L 3/502761; C12Q 1/24; G01N 15/1436; G01N 15/1459; B01L 2300/0654; B01L 2200/0652; B01L 2200/16; B01L 2300/1805; G01N 2015/149

(57) ABSTRACT

An assembly for optical preconditioning of an optically activatable biological sample comprising of cells suspended in a liquid, with a reservoir which stores the sample from which the sample are conveyed a conveying unit through a hollow channel sequentially one after the other. An illumination unit illuminates the cells contained in the sample which flow through the hollow channel at a flow rate that can be specified by the conveying unit as set by a controllable illumination intensity and illumination period and at least one of a cell analysis and sorting device in fluid communication downstream of the hollow channel.

### CROSS REFERENCE TO RELATED APPLICATIONS

Reference is made to PCT/EP2021/050252, filed Jan. 8, 2021 and to German Application No. 10 2020 200 193.6 filed Jan. 9, 2020, which are incorporated herein by reference in their entirety.

### BACKGROUND OF THE INVENTION

### Field of the Invention

The invention relates to an assembly for optically preconditioning an optically activatable biological sample.

### Description of the Prior Art

For both the quantitative measurement and the molecular characterization of biological cells, what are known as flow cytometers are used, which comprise a flow measuring cell, usually in the form of a microchannel cell which is transparent to light, through which isolated cells from a stored cell suspension flow sequentially. A light source assembly is disposed along the microchannel cell, usually in the form of at least one laser, the beam of light from which laterally irradiating or passing through each individual cell as the cells pass through a specified measuring zone along the microchannel cell. By use of suitably disposed photodetectors, both scattering components of the stimulating laser light and fluorescent light phenomena initiated by the laser beam which usually originate from fluorescent labels adhering to the cells or cell components can be detected, and are used for the simultaneous analysis of physical and molecular properties of the individual cells.

Document WO 2005/017498 A1 discloses a generic flow cytometer, which instead of the aforementioned lasers uses light emitting diodes, or LEDs for short, which irradiate the individual cells under at least one of different angles of incidence and with different wavelengths. In an appropriate manner, detectors are used to detect the scattered light components as well as the fluorescent light which is emitted due to fluorescence by the cell or from the fluorescent labels adhering to the cells.

In order to improve the reproducibility and significance of measured values which are obtained by carrying out optical cell measurements with the aid of flow cytometers, a device described in the publication WO 2017/036999 A1 for optical stimulation of an optically activatable biological sample which is stored in a sample container which is optically and thermally coupled to a temperature-controlled fluid circuit passing through a hollow channel on or in which at least one light source is disposed and which is thermally coupled thereto, which can illuminate the optically activatable biological sample stored in the sample container in a controlled manner before the sample is fed to the flow cytometer by being sucked out of the sample container.

If, there is a need for cell sorting as an alternative to or in combination with the analysis of the cells, then the cells which are usually suspended in a translucent liquid, optionally after passage through the flow cytometer, enter a microcapillary through which the isolated cells flow sequentially one after the other. Typically, the cells which flow along the microcapillary are irradiated with a laser beam and, if appropriate, stimulated into fluorescence if fluorescence labels are adhered to the cells. The scattered light and, if appropriate, the fluorescent light which is generated per cell is detected by detectors, and the detector signals therefrom form the basis of a subsequent sorting mechanism. By use of a vibrator applied to the capillary outlet, the stream of liquid is divided into small droplets which pass through an electrostatic sorting mechanism after exiting the microcapillary, by which, depending on the sorting specifications, the cells are separated into spatially separated collecting containers.

The present systems for cell analysis and cell sorting constitute tools for experiments in the field of optogenetics, from which valuable information regarding intracellular and extracellular processes can be obtained. In particular, optogenetics enables highly complex and above all rapid biochemical reactions as well as bioelectrical signal transfers within a cell to be analysed and possibly controlled. With the available use for cell analysis as well as cell sorting, however, only limited possibilities arise for controlled optical interaction with biological cells for the purposes of analysis as well as for the controlled exertion of influence on intracellular and extracellular processes.

### SUMMARY OF THE INVENTION

The invention adopts measures with which the scope for obtaining information as well as the scope for influencing or controlling intracellular as well as extracellular events can be substantially augmented compared with the currently applied and available techniques which have been described above.

Because of the complexity of intracellular structures and the associated biochemical and bioelectrical events which occur both within as well as between cells, our understanding of cells and cell processes as a whole is still incomplete. It is currently considered that one of the reasons for this is that the cells supplied to the flow cytometer are in a substantially non-specific state, that is an imprecisely defined state. This is also the case with cell sorter processes.

The underlying invention is illuminating the individual cells in a controlled manner with light of a specific quantity as well as over a specific period of time chronologically immediately before a cell analysis, which is known per se, with the aid of a flow cytometer or cell sorter, preferably on the basis of fluorescent light-based cell sorting. In this manner, specific functional events in the cells can be optically activated or deactivated in order to obtain a specific cell state in this manner which can be precisely analysed, or provides the opportunity for specific manipulations or procedures to be carried out on the cells, as will be explained below.

In accordance with the invention, an assembly is provided for optical preconditioning of an optically activatable biological sample which comprises cells suspended in a liquid. The assembly comprises a reservoir which stores the sample, from which the sample can be conveyed with a conveying unit through a hollow channel along which the cells can be conveyed sequentially one after the other, that is preferably individually and one after the other, and along which an illumination unit is disposed which illuminates the cells contained in the sample and which flow through the hollow channel at a flow rate which can be specified by the conveying unit as set by a controllable illumination intensity and illumination period. Downstream of the hollow channel, at least one of a cell analysis and sorting device is attached which is in fluid communication with the hollow channel.

The assembly in accordance with the invention, which may also be constructed and used as a modular illumination unit or illumination attachment for securing upstream of a known flow cytometer or cell sorter in the direction of flow, will be explained in more detail below with reference to the drawings.

Although a controlled illumination of a whole cell sample in connection with a known flow cytometer is known for cell analysis, this known form of illumination is not compatible with sorting flow cytometers, abbreviated to “FACS instruments” (fluorescence-activated cell sorting) (FACS is a registered trade mark from Becton, Dickinson and Company). The sample chambers for these instruments are small, built into the interior of the respective instrument and are also pressurized. Thus, it is almost impossible to use an illumination attachment for fluorescence-activated cell sorting instruments of this type. In addition, in sorting flow cytometers of this type, the entire cell sample is illuminated and then taken into the cytometer. This is not sufficient for the cell sorting procedure, especially because the time delay which is generated between the illumination and measurement/sorting of each individual cell should be identical. It would, of course, be possible to envisage an illumination of the cells along the existing capillary system of a FACS machine, but this would not enable sufficient control of the illumination period, illumination intensity or temperature of the cell sample.

The invention as described here goes around the pre-installed sample chamber of a known FACS machine and preferably uses an external conveying unit which enables the flow of the sample to be controlled, and therefore the illumination and temperature control of the cell sample along a capillary can be managed. An essential aspect in this regard is that the time interval between illumination and measurement/sorting is identical for each individual cell. In this manner, the sorting time is identical for each individual cell of the entire cell sample.

### BRIEF DESCRIPTION OF THE DRAWINGS

The invention will now be described by way of example with the aid of exemplary embodiments with reference to the drawings and without limiting the general inventive concept. In the figures:

FIG. 1 shows an assembly in accordance with the invention with multiple individual light sources;

FIG. 2 shows an assembly in accordance with the invention with at least one, preferably several light guides for illuminating the cells; and

FIG. 3 shows an assembly for positive selection of specific cells from a cell suspension.

### DETAILED DESCRIPTION OF THE INVENTION

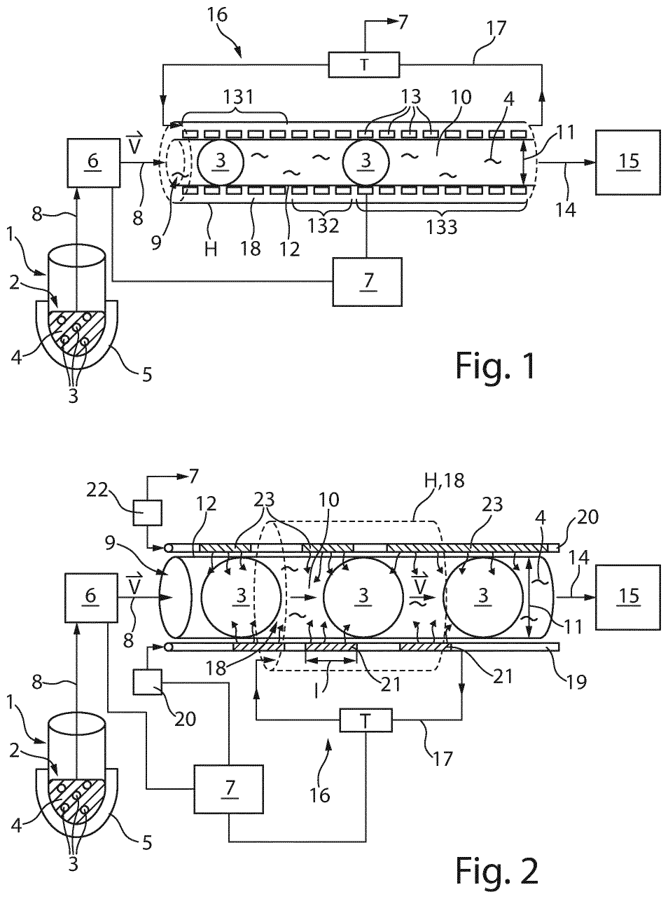

FIG. 1 shows a diagrammatic configuration of an assembly in accordance with the invention, which comprises the following components:

A reservoir 1 stores an optically activatable biological sample 2 which has biological cells 3 suspended in a translucent, that is light-permeable liquid 4. The temperature of the biological sample 2 inside the reservoir 1 can be controlled, preferably to a specifiable temperature, with the aid of a thermal unit 5.

With the aid of a fluid delivery pump 6, the displacement volume or delivery rate v of which can be controlled by at least one of controlling and regulating device 7, the biological sample 2 stored in the reservoir 1 passes via fluid lines 8 into a capillary 9 which comprises a hollow channel 10 with a capillary diameter 11 which preferably has dimensions no larger than the sum of the diameters of two cells 3 contained in the sample 2, that is the cells 3 passing along the capillary 9 preferably flow through the capillary 9 one by one, that is sequentially one after the other. Nevertheless, the dimensions of the capillary diameter may also be larger, depending on the cells present in the suspension, for example up to 200 μm, so that more than one cell can pass along the capillary next to each other, for example three to a preferred maximum of 20 cells. What is essential here is that the cell illumination for each individual cell is identical at a defined point in time, so that each cell can be transferred into a predefined optically excited state. The capillary 9 has a capillary wall 12 which is transparent to light.

In the exemplary embodiment illustrated in FIG. 1, outside the hollow channel 9, that is outside the capillary 9, individual light sources 13 are disposed, which are at least in sections along the hollow channel 10 in an axial array with respect to the hollow channel and next to each other which can be controlled individually or in groups (131, 132, 133) by use of at least one of the controlling and regulating device 7. Preferred light sources are individual illuminants or a mixture of the following illuminants: LED, laser diode, halogen lamp, gas discharge lamp, LCD, LED or OLED display unit, projector or quantum dots.

The individual light sources 13 are preferably disposed next to each other both axially as well as in the circumferential direction around the hollow channel 10. In this manner, the cells 3 which flow by the light sources 13 inside the hollow channel 10 are uniformly illuminated from all sides.

In order to enable the light input onto the individual cells 3 upon passage through the hollow channel 10 or the capillary 9 to be as individual and diverse as possible, the individual light sources 13 are gathered into groups 131, 132, 133. The light sources of each associated group 131, 132, 133 each emit a specific wavelength with a specifically definable light intensity λ131, λ132, λ133. The wavelengths λ131, λ132, λ133 as well as the associated light intensities preferably differ from one another. In principle, it is possible to select the individual light source groups 131, 132, 133, etc in a manner such that each light source group 131, 132, 133, etc contains at least one light source 13.

By the use of a large number of light sources 13 disposed in the axial extent as well as in the circumferential direction around the hollow channel 10, the light input onto the individual cells 3 can be individually specified with respect to the quantity of irradiation or the irradiation intensity as well as also with respect to the wavelength with the aid of the controlling and regulating unit 7.

The optically preconditioned cells 3 leaving the capillary 9 enter at least one of a cell analysis and sorting device 15, which is known per se, via a fluid line 14 which extends further on.

Advantageously, the capillary 9 is thermally coupled to a heat exchanger 16 which ensures that a specifiable temperature is obtained for the biological sample 2 inside the capillary 9. The heat exchanger 16 may be a Peltier element or, as can be seen in FIG. 1, is configured in the form of a temperature control unit T which is connected to a fluid circuit 17 along which a heat transfer fluid is fed, at least sections of which pass through an annular channel 18 which is radially outwardly bordered by a hollow cylinder H which radially surrounds the hollow channel 10 or the capillary 9. The heat transfer fluid which flows through the annular channel 18 is thermally coupled to the hollow channel wall or capillary wall associated with the hollow channel and therefore enables the temperature of the biological sample 2 flowing inside the hollow channel 10 to be controlled. Alternatively or in combination, the temperature control unit T may be coupled to a heat exchanger, for example in the form of an air/liquid heat exchanger, or with what are known as heat pipes.

In a further preferred embodiment, the individual light sources 13 are at least partially disposed inside the annular channel 18 so that the heat transfer fluid flows around them and they can be maintained at a uniform specifiable temperature level. In this manner, local overheating, which could be caused by the individual light sources 13, can be prevented.

With the aid of the specifiable flow rate v obtained from the delivery pump 6, the assembly in accordance with the invention enables the residence time and therefore the illumination period for the individual cells 3 inside the capillary 9 to be precisely specified. Because of the individual light sources 13, which can be operated individually or in groups in a wavelength-selective and radiation-intensity controlled manner with the aid of the controlling and regulating unit 7, the quantity of light or light intensity as well as the wavelengths of light or spectrum of wavelengths applied to the individual cells 3 can be specified individually. Thus, the biological cells 3 can be optically conditioned in a specifiable manner immediately before a cell analysis, as is known, per se, for example with the aid of a flow cytometer, or before cell sorting.

FIG. 2 shows an alternative embodiment for the implementation of the assembly in accordance with the invention for optical preconditioning of an optically activatable biological sample 2. In contrast to the assembly shown in FIG. 1, which envisages individual light sources 13 for the purpose of a controlled illumination or irradiation of the cells 3 flowing through the capillary 9. The embodiment in accordance with FIG. 2 has at least one light guide 19, for example in the form of a glass fiber, which is disposed outside the hollow channel 10 along the capillary 9. The light guide 19 is connected to a light source 20. In addition, the light guide 19 is provided with at least one light exit zone 21 laterally to its longitudinal extent which is directed onto the hollow channel 10, through which light can enter the hollow channel 10. The at least one light exit zone 21 can be produced, for example, by local roughening of the light guide 19. The roughening enables scattered light components to exit the light guide 19 laterally. The axial length 1 of the light exit zone 21 enables the illumination or illumination period for the cells 3 which pass through the capillary 9 with a specified flow rate to be specified.

The light guide 19 illustrated in FIG. 2 has a total of three light exit zones 21 which are separated from each other axially and have identical dimensions.

Optionally, at least one second light guide 20 may be disposed along the capillary 9, into which light from a light source 22 is also coupled. The light sources 20 and 22 may be identical or may provide different wavelengths. In addition, the number, arrangement and length of the light exit zones 23 along the light guide 20 may differ from the light exit zones 21 of the light guide 19. Clearly, almost any number of light guides of this type may be disposed along the capillary 9 and around its circumference.

The assembly in accordance with FIG. 2 also has a heat exchanger 16 with the associated fluid circuit 17 thereof passing through an annular channel 18 at least sections of which are disposed along the capillary 9. Optionally, the light guides 19, 20 are located inside the annular channel 18 through which the heat transfer fluid flows and in this manner, uniform temperature control is obtained.

By the controlled optical exposure in accordance with the invention of the biological cells 3 flowing in series one after the other through the capillary 9 and optionally of the dye or fluorescent substances or light-regulating particles adhered or bound to the cells 3, the cells are transformed into a defined state, forming the basis for a reproducible cell analysis, at least one of cell manipulation and cell sorting. Because of the chronologically as well as spatially defined sequence of optical cell illumination and at least one of the immediately subsequent cell analysis and cell sorting, exact optogenetic experiments and procedures may be carried out. As an alternative to cell analysis using a flow cytometer, the use of analytical instruments such as, for example, mass spectrometers or magnetic purification systems using magnetic beads, etc, is also a possibility.

Thus, this device, it is possible to verify what is known as the Kinetic Proof Reading Model (KPR) which states that T cells distinguish between endogenous and exogenous ligands by use of the differing half-lives for ligand binding to the T cell receptor (TCR). Thus, with the aid of the plant photoreceptor phytochrome B, the dynamics of ligand binding to the TCR can be selectively investigated by use of controlled illumination. By use of the reproducible optical preconditioning of the T cells to be investigated which can be obtained with the aid of the device in accordance with the invention, scientifically significant measurements for the determination of the half-life of the ligand-TCR interaction which is a decisive factor for the activation of the downstream TCR signalling can be carried out.

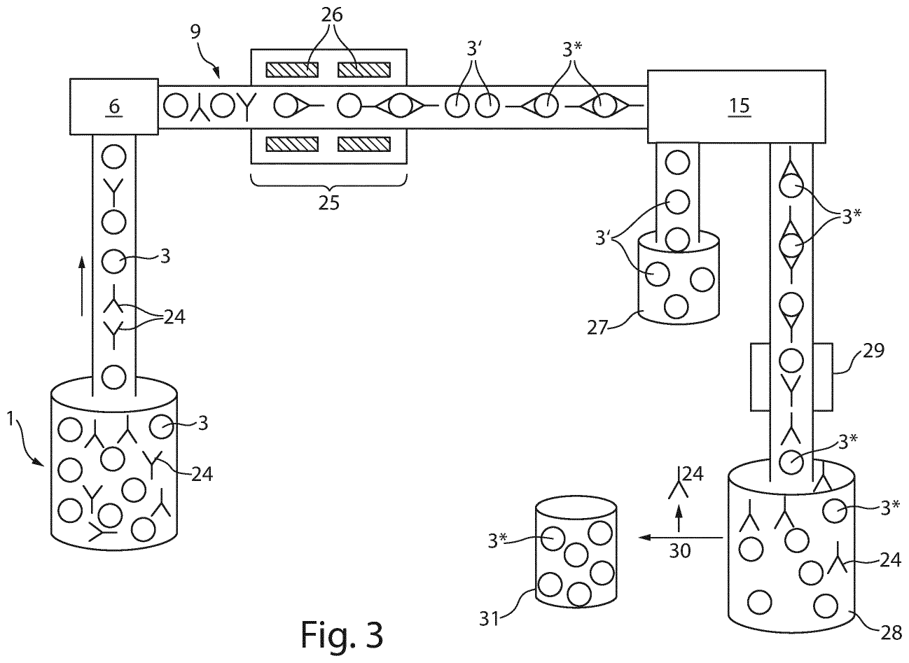

FIG. 3 shows a preferred further embodiment of the assembly in accordance with the invention with which it is possible to carry out a positive selection of cells from a mixture of cells in the form of a cell suspension, as in the case for example of blood. This possibility for positive cell selection is of particular advantage over previous methods in tumour research and cancer diagnosis.

Immune cells in particular are activated by binding of antibodies to their surface receptors and die as a result of activation. Thus, in the main, immune cell subtypes can sometimes only be negatively selected, that is a cocktail of antibodies is required which initially has to be produced and by use of which all cells with the exception of the target cells to be selected are labelled. This procedure is very costly in respect of time, procedures and techniques and only seldom leads to the isolation of a genuinely pure cell population.

With the aid of the assembly and procedure shown in FIG. 3, the cells to be positively selected are bound for a very brief period of time to a light-regulated particle without being activated thereby with the associated lethal consequences.

A cell suspension, for example in the form of a blood sample with various cells 3, for example immune cells, what are known as T cells, is situated in the reservoir 1 which is configured in an identical manner to the reservoir in FIGS. 1 and 2. The cells 3 are mixed with optically activatable particles 24, for example in the form of light-regulatable binding molecules, light-regulatable antibodies, light-regulatable single domain antibodies (nanobody) or light-regulatable adnectins (monobody).

The optically activatable particles 24 are selected in a manner such that by use of a first optical activation in the manner of a conformational change, they can be transferred from a first particle state into a second particle state in which they bind to specific cells 3* of the cell suspension to be separated. By use of a second optical activation and an associated necessary second conformational change, the optically activated particles can be transferred back into the first particle state in which they resume a non-binding state and can be released from the specific cells 3*.

The optically activatable particles 24 are selected in a manner such that by means of a first optical activation in the manner of a conformational change, they can be transferred from a first particle state into a second particle state in which they bind to specific cells 3* of the cell suspension to be separated. By means of a second optical activation and an associated necessary second conformational change, the optically activated particles can be transferred back into the first particle state in which they resume a non-binding state and can be released from the specific cells 3*.

The cell suspension stored inside the reservoir 1 is transferred by use of the conveying unit 6 along the capillary 9 into the assembly 25 for optical preconditioning.

The conveying unit 6 conveys the cell suspension with a specifiable flow rate through the hollow channel or capillary 9 along which the cell suspension is illuminated by use of the illumination unit 26 disposed inside the assembly 25 for optical preconditioning, as set by a controllable illumination intensity and illumination period. By use of this first optical activation, the optically activatable particles 24 take up the second particle state and bind to the specific cells 3*. All of the remaining cells 3′ inside the cell suspension remain in their original form.

Inside the sorting unit 15 downstream of the assembly 25 for optical preconditioning, the optically activated cell suspension undergoes optical and/or magnetically induced sorting in which the cells 3*, 3′ contained in the cell suspension are preferably sorted and separated on the basis of at least one of the quantity of emitted fluorescent light, a spectral color analysis and magnetic properties. Thus, the specific cells 3* with bound optically activated particles 24 might emit a larger quantity of fluorescent light than all of the other cells 3′, because the optically activated particles 24 are preferably coupled to a dye. Alternatively, the use of optically activatable particles 24 may be considered with magnetic particles, what are known as magnetobeads, to which the particles have been coupled. In this case, those specific cells 3* to which magnetobeads are bound via the optically activated particles can be separated by a sorting unit 15 based on magnetic force.

Downstream of the sorting unit 15 are at least two sample collection containers 27, 28. All of the specific cells 3* to be positively selected, to each of which an optically activated particle 24 has been bound, go into the sample collection container 28. A further illumination unit 29 which is disposed between the sorting unit 15 and the sample collection container 28 or is in or on the sample collection container 28, functions for the second optical activation, whereupon the particles 24 bound to the specific cells 3* are transferred back into the first particle state by use of a conformational change and are released from the specific cells 3*. All of the remaining cells 3′ are placed in the other sample collection container.

By a subsequent separation 30, for example using a centrifuge, decanter, filtration, magnetic separation, etc, the optically activatable particles 24 are separated from the specific cells 3* so that as a result, a pure population 31 of the specific cells 3* is obtained.

### LIST OF REFERENCE SIGNS

1 reservoir
2 biological sample
3 cells
3* specific cells
3′ remaining cells
4 translucent liquid
5 thermal unit
6 fluid delivery pump
7 controlling and regulating device
8 fluid line
9 capillary
10 hollow channel
11 capillary diameter
12 capillary wall
13 light source
131-133 light source group
14 fluid line
15 cell analysis device or cell sorting device
16 heat exchange unit
17 fluid circuit
18 annular channel
19 light guide
20 light source
21 light exit zone
22 light source
23 light exit zone
24 optically activatable particles
25 optical preconditioning assembly
26 illumination unit
27 sample collecting container
28 sample collecting container
29 further illumination unit
30 separation
31 pure population of specific cells
l length of light exit zone
T temperature control unit
H hollow cylinder
v delivery rate

1-21. (canceled)

22. An assembly for optical preconditioning of an optical activatable biological sample which analyzes cells suspended in the optical activatable biological sample comprising:
a reservoir for storing the activatable biological sample, a conveying unit for conveying the activatable biological sample through a hollow channel along which the cells are conveyed sequentially one after another, an illumination unit disposed along the hollow channel which illuminates the cells contained in the optical activatable sample with a controllable illumination intensity and period and the cells of the optical activatable biological sample flows through the hollow channel at a flow rate specified by the conveying unit and at least one cell analysis and sorting device in fluid communication downstream from the hollow channel for analyzing the cells suspended in the activatable biological sample.

23. The assembly as claimed in claim 22, wherein:
the hollow channel is a capillary transparent to light having a capillary diameter no larger than a sum of the diameters of two cells contained in the optical activable biological sample.

24. The assembly as claimed in claim 22, wherein:
the controllable illumination unit has first light sources disposed outside the hollow channel at least in sections along the hollow channel in an axial array relative to the hollow channel which are controllable individually or in groups.

25. The assembly as claimed in claim 24, wherein:
the controllable illumination unit has additional light sources disposed outside the hollow channel which are offset with respect to at least the first light sources in a circumferential direction around the hollow channel and in sections are disposed along the hollow channel in an axial array which are controllable individually or in groups.

26. The assembly as claimed in claim 24, wherein:
the light sources are lights or a mixture of LEDs, laser diodes, halogen lamps, gas discharge lamps, LCDs, LEDs, OLED display unit, a projector or quantum dot lights.

27. The assembly as claimed in claim 22, wherein:
the controllable illumination unit has at least one light guide disposed along the hollow channel which has at least one light exit zone directed onto the hollow channel laterally with respect to a longitudinal extension of the light guide and the light guide is optically coupled to a light source for coupling light into the light guide from the light source.

28. The assembly as claimed in claim 22, wherein:
the hollow channel is thermally coupled to a heat exchanger.

29. The assembly as claimed in claim 28, wherein:
the heat exchanger is a hollow cylinder radially surrounding the hollow channel which encloses an annular channel having a hollow channel wall and through which a temperature-controlled liquid flows which is thermally coupled to the hollow channel wall to which an optical activatable biological sample inside the hollow channel is thermally coupled.

30. The assembly as claimed in claim 29, wherein:
at least a portion of the illumination unit is disposed inside the annular channel and is thermally coupled to the temperature-controlled liquid.

31. The assembly as claimed in claim 22, comprising:
a device for controlling and regulating at least one of the conveying unit and the illumination unit in accordance with a specifiable period for irradiating the cells which pass sequentially one after another through the hollow channel with light of a constant specifiable light intensity and wavelength of a specifiable spectrum of wavelengths.

32. The assembly as claimed claim 28, comprising:
a controlling and regulating device which monitors the heat exchanger to control providing a specifiable temperature of the cells.

33. The assembly as claimed in claim 22, wherein:
the cell analysis device is a flow cytometer and the cell sorting device is a fluorescence-activated cell sorter.

34. The assembly as claimed in claim 33, wherein:
the flow cytometer has at least one pressure source which drives the conveying unit.

35. The assembly as claimed in claim 22, wherein:
the conveying unit is a membrane pump or a syringe pump.

36. The assembly as claimed in claim 22, wherein:
the cell sorting device includes a sorting mechanism with at least two downstream sample collecting containers each for receiving components of the optical activatable biological sample; and
an illumination unit for illuminating the optical activatable sample collected in at least one sample collecting container.

37. The assembly as claimed in claim 36, comprising:
an illumination unit located between the sorting mechanism and the at least one sample collecting container in or on the at least one sample collecting container.

38. A method of use of the assembly as claimed in claim 36, comprising selecting biological cells from a cell suspension containing different biological cells.

39. A method of use as claimed in claim 38, wherein:
the cell suspension is stored together with optically activatable particles as a sample in the reservoir, the optically activatable particles are transferred by a first optical activation providing a conformational change from a first particle state into a second particle state in which the optically activated particles bind to specific cells of the cell suspension and by a second optical activation providing a second conformational change back into the first particle state in which the optically activatable particles assume a non-binding state and are released from specific cells;
the optical activatable biological sample is conveyed by the conveying unit at a specifiable flow rate from the reservoir through the hollow channel which is illuminated by the illumination unit with a selected illumination intensity and selected illumination period which causes the optically activatable particles to assume a second particle state and bind to the specific cells;
the sorting device separates the specific cells to which at least one optically activated particle binds which are stored and stores the specific cells in a sample collecting container; and
an illumination unit is disposed downstream of the sorting device which illuminates the particles binding to the specific cells which returns the cells to the first particle state by a second optical activation and the particles are released from the specific cells.

40. A method as claimed in claim 39, wherein:
the optically activatable particles are one of light-regulatable binding molecules, light-regulatable antibodies, light-regulatable single domain nanobody antibodies or light-regulatable monobody adnectins.

41. A method as claimed in claim 40, comprising:
distinguishing optically activatable particles by use of at least one color characteristic, a fluorescence property or a magnetic property.

42. A method as claimed in claim 39, wherein:
the cell suspension is blood and specific cells are immune cells or T cells.
